(19) United States
(12) Patent Application Publication
(10) Pub. No.: US 2023/0352156 A1
(43) Pub. Date: Nov. 2, 2023

(54) Outcomes and Response Times for Patients in Critical Care Settings

(71) Applicant: Richard Postrel, Miami Beach, FL (US)

(72) Inventor: Richard Postrel, Miami Beach, FL (US)

(21) Appl. No.: 18/219,963

(22) Filed: Jul. 10, 2023

Related U.S. Application Data
(63) Continuation of application No. PCT/US2022/011872, filed on Jan. 10, 2022.
(60) Provisional application No. 63/135,383, filed on Jan. 8, 2021.

(51) Int. Cl. G16H 40/20; G16H 50/20; G16H 40/67; A61B 5/00; A61B 5/11

(52) U.S. Cl. CPC G16H 40/20; G16H 50/20; G16H 40/67; A61B 5/7405; A61B 5/742; A61B 5/1113; A61B 2503/045; A61B 2503/06

(57) ABSTRACT

Hospitals are stressful environments where injured and diseased persons receive healing or palliative care. Machine devices are used to monitor individual's health status and often deliver treatment. Treatment may be delivered with a goal of allowing the individual to live a relatively normal life outside the hospital, but treatments may be delivered with a goal of increasing an individual's comfort including reduction of hospital induced stress. Hospitals generally have specialized units, e.g., trauma, maternity, neonatal, pediatric, intensive car, surgery, medical, rehabilitation, neurologic, oncologic, etc. Many units may be sub-specialized, e.g., pediatric oncology, gynecologic surgery, orthopedic surgery, etc. Many units will have associated intensive care units where the more seriously ill individuals receive specialized care including intensive monitoring and varying degrees of pharmacologic and/or mechanical life or organ support.

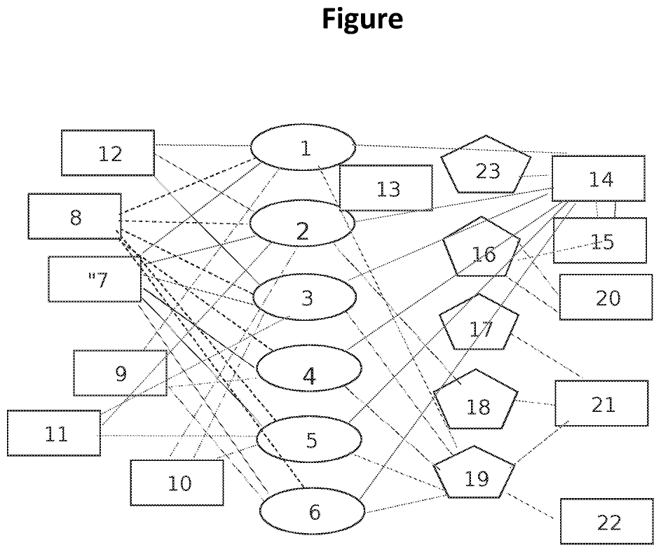

The present invention dramatically improves outcomes and survivability for all patients in critical care environments. The invention is especially useful for children of all ages and in particular for premature and neonatal infants. By eliminating extreme and continuous stress caused by very loud, attention getting, electronic sounds, alarms, flashing lights, etc., extreme stress and its resultant psychological damage is thereby minimized or eliminated. In the case of neonatal and premature children remanded to a critical care environment for extended periods of time, this invention is particularly important. Reduced stress allows the body to concentrate resources more on growing and healing, which can decrease the length of treatment in the care unit. Because these patients are deprived of bonding with their mothers in their first few days or weeks of life, this invention has the potential to reduce the probability of unintended impact caused by extreme stress in today's critical care environment. This invention also dramatically improves response times and data collection, interpretation, and management within the unit and all of its extensions. Because this time of bonding is most critical for a child's physical, mental, emotional, and social development, this invention's focus reduces long term psychological and development deficiencies caused by extreme stress at birth. This invention improves survivability in neonates and has similar reduced stress benefits for adults and older persons especially in hospital and critical care environments. Tools and methods of this invention manage and mitigate patient stress while concurrently providing a far more efficient method for communicating and recording patient needs, status, and response times.

Hospitals are stressful environments where injured, diseased and otherwise health compromised persons receive healing or palliative care. Modern machines/devices, often embracing leading edge technology are used to monitor individual's health status and often to deliver or meter treatment(s). Treatments may be delivered with a goal of allowing the individual to live a relatively normal life outside the hospital. Treatments may be delivered with a goal of increasing an individual's comfort—including reduction of hospital induced stresses. Modern hospitals or hospital systems generally have specialized units, e.g., trauma, maternity, neonatal, pediatric, intensive car, surgery, medical, rehabilitation, neurologic, oncologic, etc. Many units may be sub-specialized, e.g., pediatric oncology, gynecologic surgery, orthopedic surgery, etc. Many units will have associated intensive care units where the more seriously ill individuals receive specialized care including intensive monitoring and varying degrees of pharmacologic and/or mechanical life or organ support. In situations when patients require intensive monitoring and a high level of care, they are often admitted to an intensive care unit, a unit where a patient and devices assessing or treating each patient are extensively used and closely monitored. When a crisis situation appears in a patient, an alert is broadcast over loudspeaker throughout the patient station portion of the unit. The alert, by its nature is designed to compel attention.

The instruments of the various intensive care units are by their very nature, intensive. Devices to monitor patient health status are programmed to alert staff to any disturbance or change. Such change my indicate a situation requiring a more intense or alternative treatment. Alerts often comprise both visual and auditory stress signals. Because the alerts are invoked to redirect attention towards the alerting device, and the patient it is monitoring, by nature, the alerts are designed to raise awareness, that is to initiate an immediate or imminent response, i.e., to cause a stress directing staff to focus on the indicated need. While the alerts, as designed, are instrumental in directing or redirecting staff attention and activities, the alerts broadcast throughout the unit also affect patient health by inducing stress in all persons present. The invention may be its most impactful in a neonatal intensive care unit (NICU) setting where the patients are least able to understand the tremendous bustle in their brand new ex utero homes. The unit may be spread across many locations. For example, portions of the unit may be housed in a technology center or distributed across the cloud. Data and image displays may be remotely available and are to be considered a part of the unit when security of unit data and patient confidentiality principles, laws, rules, regulations, or the like, apply. Not all alert signals will be apparent to patients, but since they are initiated at the patient bedside, at least these portions of alerts contribute to patient stress.

In a NICU, the newborn comes into a completely foreign environment: buoyancy of the womb is lost; sounds are louder and quite different from the steady heartbeat of the mother; instead of an essentially weightless existence in the womb, gravity is impeding movement; the mother's body temperature is no longer controlling; light is newly experienced; breathing is a new skill. In the NICU, the infant may be attached to multiple devices cardiac, pulmonary, temperature (usually at multiple sites), blood pressure, endogastric, pulse oximeter, ultrasound, artificial ventilators, diaper status, etc. These devices often provide a signal indicative of their functional status, perhaps increasing volume or frequency of sound and/or changing rate, color or intensity of flashing light. And other infants in the NICU have similar devices all signaling staff of each patient's status. The various monitors produce a steady background sound. Sound is especially relevant to neonates because their retinas and visual cortex are still maturing. In full term infants at one week colors red, orange, yellow and green start to be distinguished, but there is no ability to focus. Sound is thus the more relevant of these senses for the neonate. But light distractions, especially flashing lights may still introduce significant stress.

The monitors are designed to apprise staff of normal or abnormal health events, generally by steady state continuous or repetitive sounds indicative of normal physiology. Even so, in the NICU these sounds are new and strange. Even steady state sounds are often piercing and loud and thus especially stressful to NICU patients. Older patients may be comparatively moderately stressed having experienced machines, sounds and lights in their lives and may understand to reasons for the devices. However newborns, without experience outside of the womb, find these sounds especially stressful and frightening. When monitors detect a situation where the patient needs immediate attention the device sounds an alert. The alerts are designed to command such attention, i.e., to stimulate persons responsible for the patient or malfunctioning device to immediately respond to the indicated need. The stress placed on the patient by their own monitoring devices are disturbing and extreme. But in the intensive care unit where every patient has devices that additively produce their own background sounds and alerting signals, the deleterious experience is so much the greater. Reducing a significant portion of the unnecessary stresses will allow the patient's physiology to concentrate more on growth and healing.

In an intensive care unit sound levels have been measured to rival those of a rock concert, e.g., in a range of 115 to 120 db. Sound intensities greater than 85 db are considered unhealthy. But prolonged exposures greater than 60 are commonly accepted as risking hearing loss. The human ear and auditory cortex are responsive to a range of 20 Hz to 20,000 Hz. Best sensitivity is in a range of about 2.000 to 5,000 Hz. A baby's cry has frequencies in a range from about. 1,000 to 5,000 Hz with a peak at about 3,500 Hz. The auditory and vocalization systems have co-evolved to highlight communication. For example, measured in proximity to an infant face, a screaming cry can reach 115 db, demanding a response. Artificial alarm noises, effective in drawing staff attention, may be difficult to understand by a neonate patient.

One aspect of the present invention decreases unnecessary audio stress on patients in the ICU. Reducing the clinic induced stress and patient response thereto reduces the factors the healing patient must balance and simplifies the patient healing processes. An added benefit pertains to the clinic staff. In the hubbub of the unit, the staff are bombarded with alerting sounds for all patients and equipment, not just those assigned to that staff person. The staff must filter out all the alerts and requests for which other staff members have responsibilities and focus attention on only the relevant ones. The staff responsibilities are continuously changing as patients are admitted and discharged from the unit and as other staff members start or finish shifts. Each staff member must therefore continuously update their conscious and subconscious filtering rules. This process has great potential for mistake and when a response to an alert is missed a subsequent alert must be broadcast adding still more noise for patients to stress over. However, since the filtering is inherent in the system where only relevant staff receive the alerts, the staff pay attention to all alerts they receive and are less likely to mistakenly miss calls. This improves the ability of staff to focus on caring for patients and has a secondary advantage of reducing stress on the caregivers.

The reduced stresses encountered by the patients and the more focused care can reduce time sequestered in intensive care and allow an expeditious return to normal development. Stress can affect different individuals in different aspects to different degrees. Humans have evolved to respond to or to mitigate perceived stresses. The hypothalamic-pituitary-adrenal axis is activated in stressful situations, e.g., to prepare the body to fight or flee. Many of these responses are not beneficial to normal growth and the healing process. The paraventricular nucleus (PVN) secretes corticotropin releasing factor and arginine vasopressin to stimulate a cascade of responses including release of adrenocorticotropic hormone (ACTH), cortisol, glucorticoids—epinephrine and norepinehrine, signal the liver to increase blood sugar and inhibit insulin, increase lipolysis and fatty acids in blood, increase heart rate and breathing volume, constrict airways, decrease gastro-intestinal tract activity, vasoconstriction, immunosuppression, suppression of thyroid stimulating hormone and growth hormone, decreased appetite/food intake Long term effects of stress induced glucocorticoids, at least in animals, are shown to modify neuronal structures and behaviors. Reduced stress therefore can reduce the flight or fight responses and direct the body's focus to healing.

The need to reduce stress in a NICU has been recognized. For example, tactile stresses involved with the wires leading from electrodes attached to a patient's body have been reduced in some units by using wireless communication between a sensor patch and a station data collection device. This reduces one stress on the patient by eliminating the tactile sensation felt with wire rubbing the skin and by being less restrictive to movement. One cited advantage of these wireless electrodes is reduced apprehension of patient's relatives, especially parents of newborns. As other distracting stresses are reduced remaining stresses, especially audible, take on a greater importance. While use of such wireless device to device technologies may be becoming more widespread. They are not ubiquitous. Many patients still must deal with multiple electrodes attached to their head, torso, and extremities. Every movement risks interrupting the interface between electrode and wire or the wire pulling the electrode from the skin. These occurrences result in the broadcast of a patient distress (when the signal interruption may indicate disturbance of a patient's vital function) or at least a broadcast to summon an aide or technician to restore the connection. The sounds meant to capture staff attention affect infant comfort, perception of normalcy, and sleep cycles. These and other stresses will unnecessarily affect levels of cortisol and other stress hormones in the patients and in the attentive staff. The present invention aims to improve patient outcomes by reducing stresses caused by audible alerts. By filtering alerts to deliver to individual staff only those relevant to each staffers duty assignments, staff are less distracted and can be more attentive to specific patient needs.

In a NICU environment as an example, the nurses, technicians, residents and other staff responsible for each patient serve as a team. The members of the team are assigned a channel specific to a patient. Each team member may receive input from multiple channels when the team member has multiple patient assignments. The channels may be assigned separate radio (e.g., FM) frequencies or may be keyed by a channel identifier signal, e.g., an electronic signal code that alerts the receiver assigned to the team member of incoming data information.

As an alternative or in addition to teams assembled for each patient, teams may be assigned particular classes of instrumentation. For example, a team of one or more technicians may be assigned to receive alerts from breathing monitors. The technician may be, for example a respiratory therapist or an expert in pulmonary monitor set-up, maintenance and repair. A technician may be assigned responsibility fora primary set of monitors that alert the technician to a malfunction or to successful implementation of scheduled activities. Preferably, when a primarily assigned technician is involved in responding to an alert, a second emergency alert in that area will be rechanneled to a secondary or back-up technician. The system may be programmed to rank order notifications to direct team attention to the most urgent needs. As the system functions, an artificial intelligence component may recognize strengths of individual team members their proximity to the alert and/or rank emergency calls to most efficiently respond to each patient's needs.

One significant advantage of the radio (or infrared) communication with team members is that the patients are not confused by or stressed by their machines' alerts—or the alerts from machines of other patients. These reduced stresses put upon the patients will remove one often deleterious factor from the hospital healing processes. In general, electromagnetic signaling (in the form of radio waves and/or light) delivered using any band that does not interfere with or face compromise by other electromagnetic signaling or radiation in the local environment is available for practicing the present invention. To minimize distraction between teams, communication between sending and receiving units preferably will use bands not sensed by humans, for example wifi or other radio bands, or infrared light. However, for some purposes visible light may also be advantageous, for example to point out a specific location.

Electronic communication, e.g., using wireless modems, Bluetooth, etc., have become common practice with multiple channels multiplexed for efficient data transfers. Such wireless systems or dedicated bands are available for signal and data exchange in the present invention. For example, electrodes on a patient may wirelessly communicate with a receiver with an advantage of minimizing wires that may be entangled or snagged during movements. Multiple wires connected to a loved-one may disconcert some involved in or interested in care outcomes. For these and other reasons fewer or at least less visible wiring may be desired. At an individual patient's station a unit may compile data from multiple sensors. Different sensors may be present or activated depending on the station and on the patient housed in the station. Patient characteristics including but not limited to: actual presence of a patient in the station, heartrate, blood oxygenation, EKG, EEG, temperature, torso volume, breathing rate, perspiration, urine output, catheter flow, salinity, food delivery, PICC flow, patient mass, patient movement, sound emitted, etc., may be manually entered or automatically monitored. The system may incorporate one or more devices configured to help monitor acute stress and/or algorithms may be applied to monitor outputs of several devices and provide an alert when patient stress appears to increase. Such increased stress may be indicative of a new physical stress (such as movement of the patient or health equipment on the individual that might be addressed) and/or stressful activities around the patient that might be mitigated. Changes in blood pressure, heart rate, body temperature, breathing, gut activity can indicate stress. An additional device, such as a device that monitors volatile organic compounds (VOCs) from a body may be incorporated into the system to continuously stress levels (and other health issues) with reports or alerts to the relevant staff.

The station unit may be directly or wirelessly connected to the sensing devices. A station data unit is optional but may be especially advantageous when a patient may be moved and connection to a central unit may be interrupted. The patient station unit may include recording capacity as a backup, for sending data on demand, for timed interfacing, etc. A patient station may be wirelessly or hard wire connected to a central communication unit. The concept “hard wire” is broad, inclusive of tangible physical connections that send or receive information. Devices associated with a patient station may interface through a patient station communication unit or may have a dedicated interface to a centralized processor.

Patient station units and central unit devices may be configured to conjunctively or independently to silently provide appropriate alerts and other signals. The patient station is assigned an account, a file, tagging, or other means of associating a patient with a station, the station's equipment, and staff assigned to the various treatment needs of that patient at that patient station. Such “accounts” are updated in accordance with the available staff, the patient's condition(s), the devices in use, scheduled treatments or checks, etc. Such “account” may be updated or maintained by reference to one or more other accounts or events, e.g., a staff reporting or leaving, a new device or treatment initiated, instructions from a care giver, etc. In most clinical environments a central unit receiving and compiling data from a set of patients will be advantageous for analyzing capacities and ranking requirements for immediate and/or scheduled attentions. A station unit with dedicated alert capacities may serve as a back-up should a central unit malfunction.

The system utilizes a centralized manager that coordinates functions within its unit. Each patient, each patient station, each staff member, and each device are associated to one another in a table accessible to the central processor. For example, patient A will be associated with location bed 6. Collar speaker 1216 will be associated with the staff person wearing it. Data from devices attached through bed 6 are associated with patient A. Each staff, including backup staff, with responsibilities for patient A is associated with the patient and any device at that station. Should a sensing device, that is monitoring one or more of that patient's characteristics, start to malfunction or need adjustment, only the staff members associated with the device and possibly a consultative staff member, such as a nurse or nurse practitioner, receive alerts though their associated earbud, local speaker, optical screen or projection, or headgear. Each scheduled interaction, e.g., taking vitals, changing a diaper, feeding, adjusting position, etc., associated with patient A is associated with the relevant staff member(s). A clock in the central processor will send an alert to the staff associated with the scheduled interaction. When staff report to a patient station in response to an alert or interaction request, the staff may indicate response to the central processor either manually or automatically through a proximity locator device. If a response is not documented within a predetermined time interval an alert can be transmitted to backup staff. The central processor may control a video display, for example, as a board visible to all staff and/or on a monitor such as a screen viewable by a charge nurse, charting/reporting information including, but not limited to: active alerts, locations of relevant staff or visitors (including in a waiting room), scheduled events, machine status, patient movement-vitals-etc., location of controlled substances, ambient temperature, ambient sound, etc. Such information may be in any desired format, e.g., tabular, geographic, drop down menus, clock/calendar, time lapse display, historical, predictive (based on assignment or algorithm, etc.

From a patient perspective, whenever a patient need is recognized, the staff, device, or other indicator of need, signals a central station that determines who and/or what equipment should be involved. When a patient initiates the call (e.g., pushing a button), a tactile or other confirmation of successful call may be used as feedback to reduce patient worry. Otherwise, the patient is unaware of the signal or alert and thus not stressed by it. The equipment or device instigating a call may be turned on, off, adjusted, or reset by assigned staff called to react to the alert. In each alert, all relevant staff are summoned to meet the patient's need(s). That is, an alert may be received by a single relevant individual or by a larger group. But only relevant staff will be called to respond. Other staff will not be disturbed in their tasks. The patient will not be stressed by multiple calls to gather staff necessary for the indicated response. The patients will not be stressed by multiple calls to gather staff necessary to respond to other patients.

From the perspective of a staff person, a signal indicates that a patient or device needs attention. The staff person does not have to evaluate whether the alert is for someone else. The staff knows that each alert is personally relevant and thus responds immediately to the task. The staff may signal the central controller to alert backup if the staff is encumbered responding to a previous alert. In preferred embodiments, the system is aware that that staff member has not completed their response a previous alert and automatically scans a list for the next available staff member. When a staff is unable to promptly respond, the staff knows that an alert to backup will be timely transmitted, unless the staff arrives at the patient station and thereby signals the central controller.

The central controller receives data, processes the data to determine a patient's needs, matches the need(s) with associated staff, alerts the relevant staff, monitors staff attention to the alert, transmits alert(s) to back up responders as necessary, and documents, for each patient, all events including the reason for alert, arrival of staff to address the need, and alleviation of the need. A central processor preferably receives data or information from all relevant sources, analyzes data to recognize items that require attention and then alerts appropriate staff individual(s) or group(s) without alarming the entire staff or unit. Relevant information including, but not limited to: staff on duty, capabilities of each staff (e.g., specific training or certificate), identity of staff assigned to each patient, identity of staff responsible for each device, output from the sensing devices, a schedule of staff assignments (who is there or will be there for the next scheduled task), schedule of procedures or checkups in the account of each patient, an acceptable range of result for each parameter monitored on each patient, an acceptable output range for each device, an out of service warning if a device so warns, stress indicators or change in stress indicators for a patient, activities near the patient, list of staff to contact for an out of range test result by patient and/or by device, location of staff, completion of a scheduled or on call task, etc. The central server also access an alarm or contact circuit that alerts the identified assigned staff on demand or responsible for any action, correction or sign off. In a preferred embodiment, central control may suggest an action to correct the alarm situation (e.g., replace a bulb, adjust a sensor pad, etc), to administer or change a therapy (e.g., with reference to patient history, hospital policy, treatment manual, etc.), to change diet, feeding time, patient station, etc. For patients with extended stays, in preferred embodiments, the central controller maintains patient history including result of each treatment event. When an alert is presented, the controller compares the present alert to those previously encountered. Ineffective responses are noted and those with a higher likelihood of success, especially those with historical success on that patient, are suggested to the responding staff who may not have been assigned to that patient at the previous alarm. The central controller may access guidelines, treatment protocols, or manuals to provide a responder with a step-by-step checklist.

Each alert is personal to the relevant staff rather than over a system for all to hear. The patients are not unnecessarily stressed by hearing broadcast alerts relevant to themselves or to other patients. An earbud, a collar speaker, a headset or other locally audible device may be used by individual staff to receive inputs from the central controller without stressing patients or distracting alternatively assigned staff. A headgear may be used as a substitute for an earpiece or may provide visual imaging to substitute for or to augment audio communication. As an example, earbuds may be desired, e.g., for comfort, to reduce bulk inherent in larger speaker devices, for a cleaner, more human looking appearance, but some staff may be more comfortable wearing headsets with one or more miniature speakers in, at, or proximal to one or both ears. Helmets may be used, especially when safety shields are desired. Helmets may include inserts/accessories for tactile, audio and/or visual signaling. Visual signaling may feature color codings, spatial signaling, e.g., to identify patient or to point a caregiver to a patient in need, text messaging above, alongside, or displayed within a screen.

Visual signaling may be present at a patient station. For example, the station, e.g., crib or bed, may feature signals at its base, sides or tops, if a bulb or lid is present. The base, at its simplest is a support to accept the patient, but it may include embedded or associated monitors. Sides may be used to prevent patients from edging off the support and/or for isolation. A station may feature a hood as a partial top to control air movement in the patient vicinity and may allow controlled delivery of gases, such as augmented oxygen or water vapor for the patient to breathe. The station may feature a complete top allowing total isolation of the patient. A hood or complete top may provide sufficient air flow control and may permit sampling of gases such as CO₂ and/or other gases or vapors produced by a patient.

The hood or enclosed top or lid, when available, offers an opportunity to deliver therapeutic aromas to a patient. In several embodiments, volatile organic compounds (VOCs) may be monitored from the ambient atmosphere surrounding a patient or from sensors associated with an object in the station, including, but not limited to bedding, wall, bed, clothing, banding, etc. Sensing the VOCs from the patient continuously or periodically may be incorporated alongside the alert communications system to interface with a central controller and automatically deliver one or more therapeutic aromas or to query a care giver whether such delivery should be approved. The recognition of benefits of aroma therapy is growing with substances including, but not limited to lavender, sandalwood, frankincense, parsley, davana, pine, pink lotus, jasmine, lemongrass, mugwort, sage, and sesquiterpenes as a class, etc., being applied for stress reduction and other beneficial outcomes. In future applications the system will incorporate an artificial intelligence function to enhance patient outcomes.

To provide signaling for persons distant from the patient station (e.g., a charge nurse, parent, relative, etc.) a visual communication mode may be featured on display above the patient. Communication may be from the patient unit itself and/or a central unit. Communication may use visible or invisible radiation and/or hard wiring from a sender unit. In one embodiment the patient unit may transmit a light up to a ceiling receptor unit. The light may be simply reflected off a ceiling unit e.g. to indicate a green, yellow or red status (okay, help needed soon, immediate attention required). The light may be a coded electromagnetic signal interfacing, e.g., with a ceiling or wall mounted receiver that may transmit and/or analyze the data received. Such electromagnetic signal may be redirected by reflection. The shape of a reflector unit for visual status notice or for data reception and transmission is arbitrary and a matter of functional, architectural, or aesthetic choice. A conical shape protruding downwards from the ceiling is one possible shape. A spherical or rounded bulb-like projection is another example. Such reflector may be shaped as a continuous curve or may be broken into planar or plane like surfaces. Such planes may reflect different signals in different directions, for example to obscure signals from reaching a public viewing area (to comply with HIPPA or other regulations or promises). The signal may be directed towards specific staff whose awareness of or attention to the message is desired. The system may be dynamic, possibly pivoting the reflector to be directed at a select staff. Projection to a planar surfaces may be selected with reference to geolocation devices on a person or equipment. Directing the signal may be static, e.g., where the entire back side of a room would be the target of the message or alert.

More complex patterned signaling is possible, e.g., pulsed or alternating signals changing colors, etc., to those within or outside the room who may see these signals. Preferably, signals are alternating in a smooth pattern to minimize stress that may sometimes be associated with rapidly flashing lights. For displays visible outside the room, messaging may be for more general information, e.g., sleeping or awake.

The invention features interfaces and methods that coordinate caregivers and machines to act in concert with assigned team structures to care for their patients without the conventional disconcerting and disturbing sounds. The patients are not aware of the alarming and stressful sounds. The patient's stress hormones and other responses do not complicate development and healing.

A simplified map example is provided to facilitate description of one schematic of organization in an intensive care unit. Many tasks and persons responsible for them are omitted. Many would either parallel the duties and responsibilities of other professionals or merely be responsible for different patterns of responsibilities.

This “map” shows 6 patients. This is an arbitrary number. Many installations will have more stations, but several will have fewer. In this example, a charge nurse oversees the unit or subunit. This nurse may receive only high level alerts, e.g., when an alert has been unanswered within a predetermined time for that type of event, or for select occurrences like a caregiver falling, a patient crashing, a requirement for backup power, etc. In this map, the charge nurse and the fellow have identical patient assignments. Residents are each assigned a subset of patients. Here Resident 1 is responsible for patients 1, 4 and 6; Resident 2 is responsible for patients 2, 3 and 5. A respiratory therapist is only deemed appropriate for receiving alarms from patients 2, 3 and 5. A nurse practitioner is limited to patients 1-3. The lactation consultant may not be physically present in the unit but will still receive alerts and messages relating to patients 1 and 2.

Towards the right side of the map, two aides have shared duties specifically to maintain electrode signal integrity for all six patients. Aides will have varied assignments depending on the unit. Aides may be responsible for a many tasks that may include, but not be limited to: tasking vitals, effecting or monitoring feeding, talking to and assessing patient responsiveness, cleansing patients, changing diapers, etc.

As a simple example, in a NICU, one or a plurality of sensors may reside in or in relation to a diaper. Perhaps after a preselected time, the system would alert the aides according to a plan, e.g., a closest aide, a closest aide least encumbered with other tasks, from an aide task list, etc., to direct an aide to change the diaper. In many instances one or more sensors will indicate a saturation threshold, for example, when urine volume suggests changing the diaper before the preselected time, and similarly alert attentive staff or other caregivers. In some set-ups, for some patients, a parent may be an alerted person.

Diagnostic or therapeutic instruments are arbitrarily assigned letters Y, X, W, and V. The aides are responsive to signals from Y, perhaps an indicator for a diaper in need of change, a patient with timed therapeutic or pharmacologic needs, a compromised catheter or iv delivery, etc. Technicians 1 and 2 will respond to scheduled servicing requirements for their assigned instruments or to address anomalous signals from the diagnostic and/or therapeutic devices. For example, a thermometer may indicate a need for adjustment of heating lamps or pads. A circulation therapist receives input from device V relevant to patients 1, 3, 4, 5 and 6.

A second “map” is included configured to appear more as a conventional “Figure” in a patent application.

The Figure shows 6 patients, 1, 2, 3, 4, 5, 6. This is an arbitrary number. Many installations will have more stations, but several will have fewer. In this example, a charge nurse 7 oversees the unit or subunit. This nurse may receive only high level alerts, e.g., when an alert has been unanswered within a predetermined time for that type of event, or for select occurrences like a caregiver falling, a patient crashing, a requirement for backup power, etc. In this Figure, the charge nurse and the fellow 8 have identical patient assignments. Residents 9 and 10 are each assigned a subset of patients. Here Resident 19 is responsible for patients 1, 4 and 6, 1, 4, and 6, respectively; Resident 2 10 is responsible for patients 2, 3 and 5, 2, 3, and 5, respectively. A respiratory therapist 11 is only deemed appropriate for receiving alarms from patients 2, 3 and 5, 2, 3, and 5, respectively. A nurse practitioner 12 is limited to patients 1-3, 1, 2, and 3. The lactation consultant 13 may not be physically present in the unit but will still receive alerts and messages relating to patients 1 and 2.

Towards the right side of the Figure, two aides 14 and 15 have shared duties specifically to maiietitian will analyze blood and other data from a patient and will not need to be in the unit for these types of activities. Likewise a pharmacist and discharge coordinator may serve the patient or patient's parents or guardian from a remote location.

Many of those involved in patient care are not generally present and will not be included 24/7 in the unit alert and messaging system. But when present, they can be outfitted with audio or visual communication devices, if in use. In many configurations, each relevant caregiver or technician will be wearing a speaker device in an earpiece or helmet. The alarms from each device or from another team member will feed into these e.g., earbuds rather than into the room bustle. Patients will not be bothered or upset by the continuous sounds normally present in the unit. Reduced stress improves patient outcomes over the short term with potential for reducing time in intensive care and stress of prolonged isolation from family. Stress related intellectual deficits and unhealthy behaviors in later life are mitigated.

In some embodiments the technician or caregiver may sport a helmet, wristband or other signal device. For example a caregiver with compromised hearing may see visual signals in the helmet interior or displayed on a screen that may also serve as a sanitation barrier. A wristband may provide a tactile alert directing the wearer to a screen, e.g., on the wrist, a note pad, or other location. The receiving devices are preferably equipped with geolocators that may signal the central unit of progress towards a destination or allow the central unit to choose the best recipient.

Generally, when a need is suggested or required, a local electronic sender transmits to a central processor that the determines signal insurgencies and destinations. Proper team members are sent only signals relevant to their assignments. The sound mayhem in the room is reduced allowing a calmer atmosphere and more directed attentions. If, for example, a team member is tied up with another task, the central unit may be aware of the involvement and instantaneously redirect a signal to a member assigned as back-up, may save less urgent tasks for later alert, may alert a charge nurse or other overseer, etc. Specialists not specifically assigned to the unit may receive messaging or alerts to monitor patient progress and any need to call on one or more patients in the unit. Services, such as housekeeping, pharmacy, etc.

### EXAMPLES

### Example 1

A NICU has 24 stations or beds for infant patients. The patients range in size and gestational age. But all patients present with at least one issue mandating intensive care scrutiny. The following roster of patients is merely exemplary and is not intended to be complete or exclusive. The purpose is to illustrate, by example, a complex thematic of interplays that may be required in maintaining needed therapeutics in the NICU. Other Intensive care units will have different, but many similar complexities. Three stations are vacant.

Fourteen patients require incubation with included diagnostic monitors. Two incubators are applying therapeutic hypothermia. Remaining stations whether fully configured as incubators or more open to the environment include temperature controls. Six of these patients have breathing managed using mechanical ventilation. All stations have an oxygen hood. The hood may or may not augment oxygen concentrations, but is often used to control humidity even when no oxygen is added. CPAPs are available for use at any station. The additional pressure is controlled to assist surfactants in keeping alveoli inflated. CPAPs may be used for delivering enhanced oxygen concentrations. Most patients will require timed procedures, e.g., glucose monitoring every one, two, four or six hours; electrolytes once or twice daily; diaper change set by infant size and rate of iv solution delivery; etc. All patients have PICCs inserted into a (usually leg) vein which requires periodic checking. One patient is awaiting results from a genetic test to assess a possibility of an SLC2A1 mutation

The following assignments are arbitrary and indicate merely to illustrate that many team configurations, including overlapping configurations may be assigned. Often one or two supervising neonatolgists will be present in or available to the unit. In teaching hospitals at least one neonatal fellow is directly caring for a patient. One or more pediatric residents will be on rotational assignment in the unit. Several nurses and aides will be predominant care givers. A respiratory therapist will direct or assist a technician or aide in configuring, adjusting and maintaining breathing assistance devices and oxygen delivery levels. Additional therapists may include speech, occupational or speech therapists who may have assigned hours of activities (sometimes assigned specific hours to control crowding within the unit). A dietitian will monitor a patient's nutritional needs and nutrition and often will have scheduled consultations with a speech therapist working with individual patients for suckling and other mouth behaviors. The dietitian, and speech therapist may work with a mother and lactation consultant regarding maternal milk supply (pumping and/or nursing). Not all these activities and interactions will require presence in the NICU. A dietitian will analyze blood and other data from a patient and will not need to be in the unit for these types of activities. Likewise a pharmacist and discharge coordinator may serve the patient or patient's parents or guardian from a remote location.

Patient A is housed in an incubator providing therapeutic hypothermia (controlled cooling and sedatives to maintain body temperature between 33°-34° C.). This patient has scheduled: glucose every 2 hours; arterial gases with lactate every 6 hours; electrolyte panel, CBC, platelets, PT, INR, fibrinogen, PTT, D-dimer every 12 hours; and hepatic function every 24 hours. An audible message to the “responsible nurse” says: “Karoline at station 3 is ready for her [test].” The “responsible nurse” may be specifically assigned to this patient or may be a nurse deemed most available when the system is using Artificial Intelligence. The nurse may respond or signal the system to request a back-up nurse. This request is repeated, for example, every minute for an hourly need or maybe at a 2 minute, 3 minute or longer interval for less frequent scheduled needs, until it is acknowledged as complete. When procedures require an aide or other assistance, such request may be transmitted to all suggested respondents and each may signal necessity for a backup independently. A reminder to take a patient's vitals is transmitted to a first aide according to schedule, with back-up transmissions or transmission to an alternate aide repeated until the vitals data are entered in the system. A charge nurse may see the message at a central station and/or when desired may receive signals directed at any one or more individuals. A supervisor may be alerted by audio or text signal when a certain need is not addressed within a predetermined time.

Patient B is receiving augmented oxygen in a hood. This patient has scheduled testing less detailed than patient A. Responsible staff will be signaled in a manner similar to that described above for patient A. Suddenly, a sensor indicates that patient B's breathing is not happening. An alert is sent to a nurse and the respiratory therapist. A technician is also alerted. Fortunately in this circumstance, patient B had managed to turn out of range of the microphone detecting the breathing. The summoned technician adjusted directional sensitivity with approval from the respiratory therapist and nurse.

Patient C is receiving mechanically assisted respiration. His pulse-ox meter alerts with a pO₂ at 92. A nurse, the respiratory therapist, technician, and fellow are alerted. Each responds reflexively as their earpieces direct them to station 17. The fellow contacts the neonatologist who approves adjusting the pulmonary gas pressure to keep lungs and airways more open and increases tidal volume slightly. Both agree that increasing O₂ concentration may be more risky. The technician performs the adjustments and all summoned staff go on their ways.

Patient D receives breast milk with supplemental nutrition, but requires pulmonary support. To assist lactation, the mother will allow the infant to suckle briefly with O₂ provided through the nose. Otherwise the infant is tube fed (with augmented oxygen in the bubble) or treated with CPAP. Scheduled summonses are transmitted to an aide every 2 hours for vitals and every 3 hours to position the feeding and nasal tubes and five minutes later to stop the feeding. A sensor in the diaper generally summons an aide for a change before the time scheduled change. Turning off the diaper full signal resets the countdown time to next diaper change. Alerts are sent to relevant staff with every device anomaly or unexpected symptom including, but not limited to: blood pressure, pO₂, urine acidity, blood in urine, heart rate, chest movement, etc. Only staff who may be needed to address the concern are contacted, freeing the others to concentrate on their assignments.

### Example 2

In another environment, needs are primarily indicated using light. Staff may wear a speaker, for example as an earbud, on a collar, or in an isolation mask the may be used as a backup or may function in parallel to the electromagnetic (visible light) signaling protocols.

Rather than a personalized patient relevant sound being transmitted a light is visible above the station. Preferably the light is transmitted upwards from the station to avoid misidentification of the patient in need (for example, if the patient were to be moved to an unassigned location). Position location is provided by electronic proximity indicators. A lighted cone, bulb or other shape visible from across the room will be lit with one or more colors indicating patient status. For example, a green light may signify that that patient has received all scheduled interactions and no condition requires immediate or imminent attention. An orange and blue alternating light may be a signal for both a nurse and aide to attend the patient. The light may be transmitted upwards from the station and reflected off the ceiling mounted protrusion. The light may originate in the protrusion perhaps signaled wirelessly from the station below. The ceiling unit may additionally or alternatively receive signal instruction from a central server. A board numbered with each station is also useful as an option providing similar information in a centralized location. When a board is available, one option might be to provide a red light over the station that will direct staff assigned that station to look at the board where specific immediate staffing requirements are indicated for that station. Colors may appear as numbers to indicate which specific staff should be attentive to that station. For example, a first aide may be assigned number “X47” personally; X47 would only appear when that aide was present and responsible.

The alerts, either audio or visual, and scheduled interventions are transmitted to only those in need of responding or who should be aware. Stress levels on patients are reduced with less noise. Concentration of the caregivers is more focused with improved patient outcomes. The background noise in an intensive care unit may be reduced by at least 4 or 5 fold (^{˜}20 db or greater) in many situations. Where mechanical ventilation, pneumatic cuffs to reduce risk of blood clots, staff movements, patient movements, human voice, etc., are significant, the present invention may reduce stressful background noise in the human audible range generally considered ^{˜}20 Hz to 20 kHz. Sounds in a range from about 500 Hz to 8000 Hz are considered most important for hearing tests because of the high sensitivity of the human ear in this range. Smaller ears, for example those of the average female, child or infant have narrower ear canals with a resultant reduced emphasis on lower frequencies. In accord with the present invention noise reductions of 10 db or more will be beneficial decreasing perceived sound by a factor of 2 within this frequency range or a narrower portion thereof, e.g., higher sensitivity zones of about 1000 to 5000 Hz, about 2000 to 5000 Hz, about 2000 to 4000 Hz, or 3500 Hz+/−about 500 Hz. By eliminating the broadcasts of alarms over a loudspeaker system, noise levels sound levels in the room can be significantly reduced to more generally acceptable levels.

What is claimed is:

1. A communications system comprising:
i) a central processor;
ii) at least one patient station unit; said patient station unit comprising:
a support for a patient;
a border surrounding said patient support;
at least one sensor capable of monitoring a characteristic of a patient;
an identification account identifying said patient station to said central processor;
a receiver capable of receiving signal from said central processor;
a sender capable of sending signal to said central processor; and
iii) a receiver unit configured for a responder;
said receiver unit comprising:
an identifier identifying said responder to said central processing unit;
said receiver unit programmable to correspond to at least one said patient station unit said central processor comprising:
a receiver portion configured to receive input of signal from at least one said patient station unit;
a processing portion configured to process input from said at least one patient station station unit;
said processing portion configured to process input from said at least one patient station station unit capable of accessing data to correlate said at least one patient station unit with a list matching at least one said responder to said at least one patient station unit;
a transmitter unit;
said processing portion configured to instruct said transmitter unit to transmit a call signal to said at least one responder, said call signal identifying to said at least one responder the patient station matched to said at least one responder.

2. The communication system of claim 1 further comprising a transmitter unit configured to identify said responder to said central processor and configured to transmit to said central processor arrival at a patient station unit.

3. The communication system of claim 2 wherein said transmission to said central processor is interfaced through said patient station unit.

4. The communication system of claim 1 wherein said call signal produces an audible sound.

5. The communication system of claim 1 wherein said call signal produces a visible light.

6. The communication system of claim 1 further comprising a tally board whereupon active station units are displayed and identified with corresponding station identifiers.

7. The communication system of claim 6 wherein said station identifiers are associated with a status indicator.

8. The communication system of claim 7 wherein said status indicator indicates one or more unanswered call signals.

9. The communication system of claim 7 wherein said status indicator indicates that no action is desired in the absence of at least one unanswered call signal.

10. The communication system of claim 5 wherein said visible light is visible at or above said patient station unit associated with said call signal.

11. The communication system of claim 6 wherein said status indicator comprises at least one light.

12. The communication system of claim 11 wherein said at least one light indicates one or more unanswered call signal.

13. The communication system of claim 11 wherein said at least one light indicates that no action is desired in the absence of at least one unanswered call signal.

14. The communication system of claim 2 further comprising a position location device associated with a staff, said position location device associating the staff location with the patient station unit and is programmable to notify the central processor that the staff has arrived at said patient station unit.

15. The communication system of claim 1 wherein said sensor is capable of monitoring presence of a patient.

16. The communication system of claim 1 wherein said sensor is capable of monitoring at least one characteristic selected from the group consisting of: heart rate, blood oxygenation, EKG, EEG, temperature, torso volume, breathing rate, perspiration, urine output, catheter flow, salinity, food delivery, PICC flow, patient mass, patient movement, volatile organic compounds (VOCs) emitted from the patient, and emitted sound.

17. The communication system of claim 1 wherein said at least one responder is selected from the group consisting of: nurse, charge nurse, technician, aide, fellow, resident, and therapist.

18. Use of the communication system of claim 1 wherein a patient in at least patient station exhibits less stress than said patient wherein said communication system of claim 1 is not in active use.

19. Use of the communication system of claim 1 wherein a responder assigned to at least patient station exhibits less stress than said responder wherein said communication system of claim 1 is not in active use.

20. The communication system of claim 1 further comprising sensors for volatile organic compounds in communication with said central processor.

21. The communication system of claim 20 further comprising a database of available aromas available for aromatherapy to said patient and an output from said central processor identifying at least one aroma suggested as beneficial to said patient.

22. The communication system of claim 1 wherein said sensors for volatile organic compounds in communication with said central processor apply an algorithm to data from said sensors to indicate a stress level in said patient.

23. The communication system of claim 1 wherein said central processor is configured to process information selected from the group consisting of: staff on duty, identity of staff assigned to each patient, identity of staff responsible for each device, output from a sensing device, schedule of staff assignments, schedule of procedures in the account of each patient, schedule of checks for each patient, an acceptable range of result of a parameter monitored, an acceptable output range for each device, list of contact staff for an out of range test results by patient and/or by device, an alarm or contact feature that alerts the identified assigned staff when one of said schedules indicates a task or response is due, and an app that contacts or alarms identified responsible staff according to information in a schedule, an out of range result, or a device malfunction.

24. The communication system of claim 23 wherein said central processor is equipped with an artificial intelligence function that analyzes the data from each patient, compares said data to historical data from said each patient, ranks responses according to beneficial outcome, and provides a responding staff with recommendation(s) relating to earlier response(s).

25. The communication system of claim 24, wherein said ranking responses according to beneficial outcomes includes information from hospital guidelines, treatment protocols, medical journals, or manuals to provide a responder with a step-by-step checklist.

26. The communication system of claim 23 wherein said central processor is equipped with an artificial intelligence function that analyzes the data from a patient, compares said data from said patient with additional information in hospital guidelines, treatment protocols, medical journals, or manuals to provide a responder with at least one recommendation relating to said patient data and said additional information.

27. The communication system of claim 1 wherein said central processor comprises an interface that communicates with a network of remote servers that operates as a single ecosystem.

28. A patient care unit comprising the communication system of claim 1.
